(19) United States
(12) Patent Application Publication
(10) Pub. No.: US 2003/0207268 A1
(43) Pub. Date: Nov. 6, 2003

(54) Methods for the diagnosis and prognosis of acute leukemias

(76) Inventors: Evan W. Steeg, Kingston (CA); Stephen R. Misener, Perth (CA); Edward S. Willis, Nepean (CA); Gary E. Anderson, Kingston (CA); Roderick J. Dunne, Kingston (CA); Marc A. Lepage, Kingston (CA)

Correspondence Address: STERNE, KESSLER, GOLDSTEIN & FOX PLLC, 1100 NEW YORK AVENUE, N.W., WASHINGTON, DC, 20005, US

(21) Appl. No.: 09/938,544

(22) Filed: Aug. 27, 2001

Related U.S. Application Data
(63) Continuation of application No. 09/726,427, filed on Dec. 1, 2000, now abandoned.
(60) Provisional application No. 60/168,625, filed on Dec. 3, 1999.

(51) Int. Cl. C12Q 1/68

(52) U.S. Cl. 435/6

(57) ABSTRACT

The present invention relates to the diagnosis of the distinction between acute lymphoblastic leukemia (ALL) and acute myeloid leukemia (AML) and prognosis of AML. Disclosed is a means to diagnose the distinction between ALL and AML employing measurement of the abundance of the nucleic acid or protein products of small combinations (two, three or more) of particular human genes. The invention further describes the use of the measurement of the abundance of the nucleic acid or protein product of two human genes for prognostic indication in AML. The invention also relates to therapies targeted at these indicator genes, and the screening of drugs for cancer that target these indicator genes or their protein products.

### CROSS-REFERENCE TO RELATED APPLICATIONS

The present application claims priority benefit of U.S. Appl. No. 60/168,625, filed Dec. 3, 1999, the entire disclosure of which is incorporated by reference herein.

### BACKGROUND OF THE INVENTION

1. Field of the Invention

The present invention relates to methods of classifying acute leukemias. More particularly, the invention relates to methods of distinguishing acute myeloid leukemia (AML) from acute lymphoblastic leukemia (ALL) by measuring the nucleic acid levels or gene product (protein) levels of small combinations (two, three or more) of particular human genes. The invention is also useful as a prognostic indicator in AML.

2. Related Art

A major challenge of cancer treatment has been to target specific therapies to pathogenically distinct tumor types, to maximize efficacy and minimize toxicity. Improvements in cancer classification have thus been central to advances in cancer treatment.

Cancer classification has been based primarily on morphological appearance of the tumor, but this has serious limitations. Tumors with similar histopathological appearance can follow significantly different clinical courses and show different responses to therapy. In a few cases, such clinical heterogeneity has been explained by dividing morphologically similar tumors into subtypes with distinct pathogeneses. Key examples include the subdivision of acute leukemias, non-Hodgkin's lymphomas, and of childhood “small round blue cell tumors” into neuroblastomas, rhabdomyosarcoma, Ewing's sarcoma, and other types. For many more tumors, however, important subclasses are likely to exist but have yet to be defined by molecular markers. For example, prostate cancers of identical grade can have widely variable clinical courses, from indolence over decades to explosive growth causing rapid patient death.

Cancer classification has been difficult in part because it has historically relied on specific biological insights, rather than systematic and unbiased approaches for recognizing tumor subtypes.

Acute leukemia is a disease of the leukocytes and their precursors. It is characterized by the appearance of immature, abnormal cells in the bone marrow and peripheral blood and frequently in the liver, spleen, lymph nodes, and other parenchymatous organs. The clinical picture is marked by the effects of anemia, which is usually severe (fatigue, malaise), an absence of functioning granulocytes (proneness to infection and inflammation), and thrombocytopenia (hemorrhagic diathesis). The spleen and liver usually are moderately enlarged, while enlarged lymph nodes are seen mainly in the pediatric lymphoblastic leukemias. Fever and a very high ESR complete the picture. Leukocyte counts vary greatly in the acute leukemias. About one-fourth to one-third of cases begin with a low white blood count (sub- or aleukemic leukemia), while about half show some degree of leukocytosis. Mature granulocytes may still be found in the peripheral blood in addition to abnormal forms. The coexistence of immature and mature cell forms is termed “hiatus leucaemicus.” The leukocytopenic forms are the most difficult to differentiate from aplastic anemias, pancytopenias, and the myelodysplastic syndromes. Bone marrow aspiration is usually necessary to establish a diagnosis. Aspirated marrow is found to be permeated by abnormal cells (paramyeloblasts, paraleukoblasts, nonclassifiable cells (N.C.), leukemic cells, blasts, etc.) with little or no evidence of normal hematopoiesis.

The acute leukemias have traditionally been classified according to morphologic, cytochemical, and/or immunologic criteria. An overview of acute leukemia classification can be found in the “Atlas of Acute Leukemia” available on the world wide web at www.meds.com/leukemia/atlas/acute-leukemia.html.

As a brief historical review, the classification of acute leukemias began with the observation of variability in clinical outcome (Farber, S., et al., N. Engl. J. Med. 238:787 (1948)) and subtle differences in nuclear morphology (Forkner, C. E., Leukemia and Allied Disorders, MacMillan, New York (1938); Frei, E., et al., Blood 18:431 (1961); Medical Research Council, Br. Med. J. 1:7 (1963)). Enzyme-based histochemical analysis were introduced in the 1960s to demonstrate that some leukemias were periodic acid-Schiff positive, whereas others were myeloperoxidase positive (Quaglino, D., and Hayhoe, F. G. J., J. Pathol 78:521 (1959); Bennett, J. M., Dutcher, T. F., Blood 33:341 (1969); Graham, R. C., et al., J. Histochem, Cytochem 13:150 (1965)). This provided the first basis for classification of acute leukemias into those arising from lymphoid precursors (acute lymphoblastic leukemia, ALL) or from myeloid precursors (acute myeloid leukemia, AML). This classification was further solidified by the development in the 1970s of antibodies recognizing either lymphoid or myeloid cell surface molecules (Tsukimoto, I., et al., N. Eng. J. Med. 294:245 (1976); Schlossman, S. F., et al, Proc. Natl. Acad. Sci. U.S.A. 73:1288 (1976); Roper, M., et al., Blood 61:830 (1983); Sallan, B. S. E., et al., Blood 55:395 (1980); Pesando, J. M., et al., Blood 54:1240 (1979)). Most recently, particular subtypes of acute leukemia have been found to be associated with specific chromosomal translocations—for example, the t(12;21)(p13;q22) translocation occurs in 25% of patients with ALL, whereas the t(8;21)(q22;q22) occurs in 15% of patients with AML (Golub, T. R., et al., Proc. Natl. Acad. Sci. U.S.A. 92:4917 (1995); McLean, T. W., et al., Blood 88:4252 (1996); Shurtleff, S. A., et al., Leukemia 9:1985 (1995); Romana, S. P., et al., Blood 86:4263 (1995); Rowley, J. D., Ann. Genet. 16:109 (1973)).

Although the distinction between AML and ALL has been well-established, no single test is currently sufficient to establish the diagnosis. Rather, current clinical practice involves an experienced hematopathologist's interpretation of the tumor's morphology, histochemistry, immunophenotyping, and cytogenetic analysis, each performed in a separate, highly specialized laboratory. Although usually accurate, leukemia classification remains imperfect and errors do occur.

Distinguishing ALL from AML is critical for successful treatment; chemotherapy regimens for ALL generally contain corticosteroids, vincristine, methotrexate, and L-asparaginase, whereas most AML regimens rely on a backbone of daunorubicin and cytarabine (Pui, C. H., and Evans, W. E., N. Engl. J. Med. 339:605 (1998); Bishop, J. F., Med. J. Aust. 170:39 (1999); Stone, R. M. and Mayer, R. J., Hematol. Oncol. Clin. N. Am. 7:47 (1993)). Although remission can be achieved using ALL therapy for AML (and vice versa), cure rates are markedly diminished, and unwarranted toxicities are encountered.

Recently, Golub, T. R., et al., Science 286: 531-537 (October1999), have reported on a cancer classification scheme for AML and ALL based on the gene expression monitoring of 50 human genes. Although the 50-gene predictor approach for diagnosing AML versus ALL fared well in validation studies, the Golub et al. report noted that the average prediction strength was lower for samples from a different laboratory, thus emphasizing the importance of standardizing sample preparation. Further, the application of 50 genes for AML-ALL class distinction may not be desirable for a clinical setting. A method/tool employing fewer indicator genes/gene products than used by Golub et al. would provide increased ease, increased speed, and reduced cost. Potential for human error (misidentification) could be reduced. Reliance on expert, trained interpretation of data could also be reduced. Rapid diagnosis based on the non-random correlations (“diagnostic signatures” or “fingerprints”) according to the invention described below thus would produce enormous benefit. Clearly, there is a continued need for simpler and less costly objective cancer classification approaches, especially for the classification of acute leukemias.

### SUMMARY OF THE INVENTION

The inventors have discovered that measuring the levels of small combinations (two, three or more) of particular human genes (in terms of nucleic acid or protein levels) can be used to distinguish AML from ALL. Accordingly, the present invention overcomes the disadvantages of the prior art by providing a method for diagnosing leukemia by measuring the levels of a lesser number of genes than provided in the art.

The invention also provides a preferred embodiment of the foregoing method wherein the human genes used to diagnose are LYN V-yes-1 Yamaguchi sarcoma viral related oncogene homolog, PPGB Protective protein for beta-galactosidase, and Zyxin.

In the most preferred embodiment of the foregoing method, the genes used to diagnose are: leukotriene C4 synthase (LTC4S) gene and Zyxin.

The invention also provides a very particularly preferred embodiment of the foregoing methods, wherein the level of gene expression is measured using a DNA microchip.

The present invention also provides an embodiment, whereby the measurement of at least two human genes is used as a prognostic indicator of AML.

The present invention also provides a kit for diagnosis or prognosis of leukemia.

The invention also relates to therapies targeted at the indicator genes described herein, as well as the screening of drugs for cancer that target these indicator genes or their protein products.

It is to be understood that both the foregoing general description and the following detailed description are exemplary and explanatory and are intended to provide further explanation of the invention as claimed.

### DETAILED DESCRIPTION OF THE PREFERRED EMBODIMENTS

The inventors have discovered that measurement of the levels of only a few human genes (nucleic acid levels or protein levels) can be used to distinguish AML from ALL. By “nucleic acid” is intended RNA or DNA, preferably mRNA or cDNA derived therefrom. Accordingly, the present invention overcomes the disadvantages of the prior art such as Golub et al. (1999), supra, by providing a method for diagnosing and classifying acute leukemia by measuring the expression levels of a lesser number of genes or gene products.

The names of the genes useful in diagnosis and/or prognosis described herein are as designated by Affymetrix and Golub et al., and, according to them, correspond, as indicated in Appendix B, to particular GenBank entries.

The invention also provides a preferred embodiment of the foregoing method wherein the human genes used to diagnose are: LYN V-yes-1 Yamaguchi sarcoma viral related oncogene homolog, PPGB Protective protein for beta-galactosidase, and Zyxin. These gene names are as assigned by Affymetrix and Golub et al., and according to them, correspond to GenBank Accession Nos. M16038_at, M22960_at, and X95735_at, respectively.

In the most preferred embodiment of the foregoing method, the genes used to diagnose are: leukotriene C4 synthase (LTC4S) gene and Zyxin. These gene names are as assigned by Affymetrix and Golub et al., according to them, correspond to GenBank Accession Nos. U50136_ma1_at, and X95735_at. respectively.

Other embodiments employ other csets which are identified in Appendix A.

It is expected that, for certain csets, an inverse pattern of gene expression of ALL markers, as disclosed herein, would correlate with AML diagnosis. Likewise, an inverse pattern of gene expression of AML markers, as disclosed herein, would correlate with ALL diagnosis.

The invention also provides a very particularly preferred embodiment of the foregoing methods, wherein the level of gene expression is measured using a DNA microchip.

The present invention also provides an embodiment, whereby the measurement of small combinations (two, three or more) of particular human genes is used as a prognostic indicator of AML.

The present invention also provides a kit for diagnosis or prognosis of leukemia.

Gene expression data from the database http://waldo.wi.mit.edu/MPR/data_set_ALL_AML.html (which was made publicly available on Oct. 15, 1999) was analyzed as described below. Per Golub et al., Science 286: 531-537 (Oct. 15, 1999), incorporated herein by reference, the database contains the levels of expression of each of 7129 genes for each of 72 leukemia samples, which levels were determined using Affymetrix genechip technology. The samples were classified by Golub et al. as either acute myeloid leukemia (AML) or acute lymphoblastic leukemia (ALL) and this information is also included in the database. The database further includes clinical data on 15 individual acute myeloid leukemia (AML) samples, with respect to treatment success or failure.

The present inventors set out to detect signal(s) from the noise in the huge data set, i.e., to identify previously unrecognized correlated gene expression levels of groups of genes. To this end, the raw gene expression data was used in that form or processed using a standard data normalization technique (linear transformation followed by logarithm). Next, the expression levels for each gene were subjected to one of two standard data clustering techniques (“K means” as practiced by those skilled in the art or “Mutual nearest neighbors” as described in Jarvis, R. A. and Patrick, E. A., IEE Trans. Computers C-22:1025-1034 (1973)). Such pre-processing made the subsequent identification of correlations more convenient. “Clustering”, as it is commonly held in the art, refers to methods for grouping “objects” of a system based on some similarity measure. The set of values in the system being analyzed is replaced by another, smaller set of values in a way that reflects the original distribution according to a chosen distance metric. In effect, clustering forces objects into likely groups. Here, the objects were the various experimentally determined levels of expression of a particular gene. The clustering algorithm provided grouping of the expression level for each gene into classes, as set forth in Appendix A. For example, referring to line 3 of Appendix A (cset 2), experimentally determined expression levels of gene 1745 may be grouped into low (A, mean=429.4) and high (B, mean=2211.2). In contrast, the grouping of expression levels for gene 3320, line 1 (cset1) was into three classes, low (A, mean=923.6), medium (B, mean=2405.8), and high (C, mean=3496.8). (See Appendix B for the Affymetrix and Golub et al. assigned name corresponding to the gene numbers employed herein. For example, gene 1745 corresponds to Affymetrix and Golub et al. name LYN V-yes-1 Yamaguchi sarcoma viral related oncogene homolog).

Next, the pre-processed data was subjected to a variant of the “coincidence detection” method described in International Patent Publication No. WO 98/43182, published Oct. 1, 1998 (incorporated herein by reference). This method provides the identification of features which are sets of attributes (values) that co-occur more often than by random assortment and, accordingly, the identification of inherent, often unexpected features of a system. Unlike other approaches to such identification, the number of members of the identified set is not chosen prior to application of the method. That is, some approaches seek correlations between pairs of attributes (binary or 2-ary correlations). Instead, the coincidence detection method does not impose that k (as in k-ary correlations) be any specific number. Rather, the patterns inherent in the system are uncovered. As employed herein, “objects” were samples and “attributes” were gene expression values for particular genes, the ALL versus AML diagnosis, and treatment outcome for some AML samples. The high-order correlations (“coincidence sets” or “csets”) discovered by the coincidence detection method were further filtered and sorted by application of another correlation test. Matthews correlation (also known as “Four-point Correlation”) is a standard, known, though less commonly-used variant of the standard Pearson correlation measure, especially suited for discrete (as opposed to continuous) data. In this case, a Matthews correlation was calculated between (1) particular correlated gene expression values, considered together for the k genes in the particular cset and (2) the attribute corresponding to AML or ALL diagnosis, and the csets were sorted from highest to lowest Matthews correlation. These Matthews-tagged csets may be interpreted as “rules” relating particular genes and their expression-value ranges to diagnosis or prognosis. A plausible English interpretation of such a discovered rule (see second cset in appendix A) might be, for example,

“Gene 1745 has expression level A (LOW relative to a control, that is, value closest to the calculated cluster mean of 429 for this gene in one analysis performed and described herein) AND Gene 1829 has value B (LOW relative to a control) AND Gene 4847 has value A (LOW relative to a control) IF AND ONLY IF the patient has leukemia type ALL (with probability based on Matthews correlation of 0.9077).”

Appendix A shows csets obtained from clustered raw data and from clustered log normalized data. Where the same cset appears more than once in Appendix A, this derives from results of multiple experimental runs (different clustering techniques).

Thus, using these techniques, the present inventors discovered small combinations of genes that provide a diagnostic indication of acute leukemia subtype. In addition, they also discovered small combinations of genes that provide a prognostic indication for AML.

As these results indicate dependence of leukemia subtype on clustered gene expression levels, they are also indicative of dependence of the subtype on unclustered (or raw) gene expression levels. This latter relationship was confirmed by the present inventors using supervised learning techniques (artificial neural networks, decision trees, etc.) as known by those skilled in the art and as described in Mitchell, T. B., in: Machine Learning, chapters 3 and 4. McGraw-Hill (1997). The expression levels, for the genes discovered by the coincidence detection method, were given (in raw form, that is, unnormalized and unclustered) to the supervised learning agent and the subtype of leukemia (AML versus ALL) was predicted. The training of a neural network, and the use of a trained neural network for prediction or classification, is well known to those skilled in the art.

Genes correlated with specific disease subtypes are likely to have a specific role in the disease condition, and hence are valuable targets for new therapeutics.

Genes correlated with disease prognosis are likely to have a specific role in the disease condition, and hence are valuable targets for new therapeutics. Accordingly, the invention provides methods of screening for drugs that modulate (enhance or inhibit) expression of genes in the csets, or modulate (enhance or inhibit) the activity of products of such genes.

For example, screening methods for identifying compounds capable of treating acute leukemia include contacting cells with the candidate compound, measuring gene expression, and comparing the gene expression of a particular cset to a standard expression of a particular cset, the standard being assayed when contact is made in absence of the candidate compound; whereby, a difference in gene expression indicated that the compound may be useful for treating particular subtypes of acute leukemia.

High-order correlated genes are likely to play a synergistic or antagonistic role in the disease condition, and are likely to reveal important pathways involved in the disease process.

Certain tissues in mammals with leukemia express enhanced and/or diminished levels of certain proteins and mRNA when compared to a corresponding “standard” mammal, i.e., a mammal of the same species not having the leukemia. Further, it is believed that enhanced levels of certain proteins and mRNA can be detected in certain body fluids (e.g., sera, plasma, urine, and spinal fluid) from mammals with leukemia when compared to body fluids from mammals of the same species not having the leukemia. Thus, the invention provides a diagnostic method useful during leukemia diagnosis, which involves assaying the expression level of a gene or set of genes in mammalian cells or body fluid and comparing the gene expression level with a standard gene expression level, whereby a difference in the gene expression level over the standard is indicative of a specific type of leukemia. In the working examples disclosed herein, comparison was made between ALL and AML samples.

Where a leukemia diagnosis has already been made according to conventional methods, the present invention is useful for confirmation thereof and as a prognostic indicator, where patients exhibiting differing gene expression will experience a better or worse clinical outcome relative to other patients.

By “assaying the level of the gene expression” is intended qualitatively or quantitatively measuring or estimating the level of the protein or the level of the mRNA encoding the protein in a first biological sample either directly (e.g., by determining or estimating absolute protein level or mRNA level) or relatively (e.g., by comparing to the protein level or mRNA level in a second biological sample).

Preferably, the protein level or mRNA level in the first biological sample is measured or estimated and compared to a standard protein level or mRNA level (e.g., ALL sample v. AML sample), the standard being taken from a second biological sample obtained from an individual not having that leukemia. As will be appreciated in the art, once a standard protein level or mRNA level is known, it can be used repeatedly as a standard for comparison.

By “biological sample” is intended any biological sample obtained from an individual, cell line, tissue culture, or other source which contains protein or mRNA. Biological samples include mammalian body fluids (such as sera, plasma, urine, synovial fluid and spinal fluid) which contain secreted mature protein, and ovarian, prostate, heart, placenta, pancreas liver, spleen, lung, breast and umbilical tissue.

The present invention is useful for detecting acute leukemia in mammals. Preferred mammals include monkeys, apes, cats, dogs, cows, pigs, horses, rabbits and humans. Particularly preferred are humans.

In order to detect gene expression, total cellular RNA can be isolated from a biological sample using the single-step guanidinium-thiocyanate-phenol-chloroform method described in Chomczynski and Sacchi, Anal. Biochem. 162:156-159 (1987). Levels of mRNA encoding the protein (or cDNA prepared from such mRNA) are then assayed using any appropriate method. These include Northern blot analysis (Harada et al., Cell 63:303-312 (1990)), S1 nuclease mapping (Fujita et al., Cell 49:357-367 (1987)), the polymerase chain reaction (PCR), reverse transcription in combination with the polymerase chain reaction (RT-PCR) (Makino et al., Technique 2:295-301 (1990)), and reverse transcription in combination with the ligase chain reaction (RT-LCR).

Protein levels may be determined by assaying enzymatic activity of the protein. This is especially useful when screening potentially useful therapeutic drugs that affect protein activity.

Assaying protein levels in a biological sample can also be performed using antibody-based techniques. For example, protein expression in tissues can be studied with classical immunohistological methods (Jalkanen, M., et al., J. Cell. Biol. 101:976-985(1985); Jalkanen, M., et al., J. Cell. Biol. 105:3087-3096 (1987)). This is useful when screening drugs as potential therapeutics that affect gene expression.

Other antibody-based methods useful for detecting protein gene expression include immunoassays, such as the enzyme linked immunosorbent assay (ELISA) and the radioimmunoassay (RIA).

Suitable labels are known in the art and include enzyme labels, such as, glucose oxidase, horseradish peroxidase and alkaline phosphatase; radioisotopes, such as iodine (¹²⁵I, ¹²¹I), carbon (¹⁴C), sulfur (³⁵S), tritium (³H), indium (¹¹²In), and technetium (^{99m}Tc); fluorescent labels, such as fluorescein and rhodamine; and biotin.

In a preferred embodiment, gene expression is measured using a DNA microchip, as described below in Example 3. DNA microchips are described in U.S. Pat. Nos. 5,744,305; 5,424,186; 5,412,087; 5,489,678; 5,889,165; 5,753,788; and 5,744,101; and WO 98/12559; and Harris, Exp. Opin. Ther. Patents 5:469-476 (1995). DNA microchips contain oligonucleotide probes affixed to a solid substrate, and are useful for screening a large number of samples for gene expression.

The present invention also further includes kits for diagnosing subtypes of acute leukemia, comprising a means for measuring gene expression of each gene of a cset which is herein disclosed as being correlated with a subtype of leukemia, wherein said means are within a container. In one embodiment, a kit is provided which comprises a means for measuring gene expression of LYN V-yes-1 Yamaguchi sarcoma viral related oncogene homolog, a means for measuring gene expression of PPGB Protective protein for beta-galactosidase, and a means for measuring gene expression of Zyxin. In one embodiment, the means for measuring gene expression is a DNA microchip which contains probes specific for the target gene(s). In another embodiment, the means for measuring gene expression is an antibody specific for the protein of interest. Other means for measuring gene expression are well known in the art.

The invention also relates to therapies targeted at these indicator genes, as well as the screening of drugs for cancer that target these indicator genes or their protein products.

Having generally described the invention, the same will be more readily understood by reference to the following examples, which are provided by way of illustration and are not intended as limiting.

### EXAMPLES

### Example 1

Those skilled in the art can, by the exercise of ordinary skill, measure the mRNA or protein level for each of the two, three or more (preferably two to six) genes in a correlated set discovered to be diagnostic for leukemia subtype and, in reference to a standard, classify new cases of leukemia with respect to subtype. Such an analysis would be highly amenable to modem diagnostic “chip” technology and suitable for incorporation into a bedside diagnostic device.

For example, in reference to Appendix A, page a, cset 2, the expression level of Affymetrix designated genes LYN V-yes-1 Yamaguchi sarcoma viral related oncogene homolog (GenBank Accession #M16038), PPGB Protective protein for beta-galactosidase (galactosialidosis) (GenBank Accession #M22960), and Zyxin (GenBank Accession #X95735) is diagnostic of ALL. In this case, diagnosis of ALL can be made if the relative expression level of each of these genes is low. Similarly, other csets in Appendix A provide diagnostic gene “signatures” or “fingerprints” of similar value.

### Example 2

Those skilled in the art can measure the mRNA or protein level for each of the genes in a correlated set discovered to be a prognostic indicator for AML, and in reference to a standard, predict patient response to treatment. Such an analysis could be extremely valuable in designating patients as unlikely to respond to conventional therapy, and hence targeting them for more intensive or more experimental procedures.

For example, in reference to Appendix C, cset 2, the expression level of genes 1436 and 3847 (Affymetrix designated genes POU3F1 POU domain, class 3, transcription factor 1, GenBank Accession No. L26494_at; and GB DEF=homeodomain protein HoxA9 mRNA, GenBank Accession No. U82759_at, respectively) is a prognostic indicator for AML. In this case, AML prognosis is good if the relative expression level of these genes is medium-high and high, respectively.

### Example 3

Total RNA is extracted from tissue samples of a patient with leukemia, and cDNA is prepared using methods well known in the art. Double-stranded DNA is made from the cDNA. The double-stranded cDNA is transcribed using the Ambion T7 MegaScript Kit. The cRNA made from the in vitro-translation of the double-stranded cDNA is fragmented by adding 15 μg cRNA to 0.2 vol of 5× fragmentation buffer and storing at 95° C. for 35 minutes. The fragmented cRNA is then added to 3 uL 5 nM Control Oligonucleotide B2 (Final concentration: 50 pM)(Affymetrix); 3 uL 10 mg/ml Herring Sperm DNA (Final concentration: 0.1 mg/ml)(Promega/Fisher Scientific); 3 uL 50 mg/ml Acetylated BSA (Final concentration: 0.5 mg/ml)(Gibco BRL Life Technologies); 150 ul 2× MES Hybridization Buffer (Final concentration: 1×). The volume is adjusted with DEPC H₂O to 300 uL total volume.

A 12× MES Stock buffer is prepared: 70.4 g MES free acid monohydrate (Final concentration: 1.22 M MES)(Sigma Chemicals); 193.3 g MES sodium salt (Final concentration: 0.89M [Na+])(Sigma Chemicals); 800 ml DEPC H₂O; the volume is brought up with water to 1000 ml. pH should be between 6.5 and 6.7.

A DNA microchip, containing probes for LYN V-yes-1 Yamaguchi sarcoma viral related oncogene homolog, PPGB Protective protein for beta-galactosidase, and Zyxin, is prepared using, for example, the methods described in U.S. Pat. No. 5,744,305, which is herein incorporated by reference. The microchip is equilibrated to room temperature just before use. The chips are prewet with 200 uL of 1× MES Hybridization buffer at 45° C. for 10-20 minutes, 60 RPM. The fragmented cRNA is heated at 99° C. for 5 minutes and cooled at 45° C. for 5 minutes, then spun at maximum speed for 5 minutes. The 1× MES hybridization buffer is removed from chips, and 200 μl fragmented cRNA is added to each chip. The chips are incubated at 45° C., 60 RPM for 16 hours. After 16 hour hybridization, the cRNA is removed from the chip and stored at −80° C.

For each chip: 1200 uL SAPE (Streptavidin Phycoerythrin) Solution is prepared, using 600 uL 2× Stain buffer; 120 uL 20 mg/mL Acetylated BSA (Final concentration: 2 mg/mL); 12 uL 1 mg/mL SAPE (Final Concentration: 10 ug/mL)(Molecular Probes); 468 uL DEPC H₂O. 600 uL Antibody Solution is prepared, using: 300 uL 2× Stain Buffer; 60 uL 20 mg/mL Acetylated BSA (Final concentration: 2 mg/mL); 30 uL goat serum (Final concentration: 5%)(Sigma Chemical); 3.6 uL 0.5 mg/mL biotinylated anti-streptavidin antibody (Final concentration: 3 ug/mL)(Vector Laboratories); and 206.4 uL DEPC H₂O.

2× Stain buffer is prepared using 41.7 ml 12× MES Stock Buffer (Final concentration: 100 mM MES); 92.5 ml 5 M NaCl (Final concentration: 1 M [Na+]); 2.5 ml 10% Tween 20 (Final concentration: 0.05% Tween); 112.8 ml DEPC H₂O; filtering through a 0.2 um filter; after filtering, add 0.5 ml of 5% Antifoam.

Hybridization is performed using the Affymetrix GeneChip© Fluidics Station 400 at 10 cycles of 2 mixes per cycle with Non-Stringent Wash Buffer at 25° C.; 4 cycles of 15 mixes per cycle with Stringent Wash Buffer at 50° C.; probe is stained with the first aliquot of the SAPE solution for 10 minutes at 25° C.; 10 cycles of 4 mixes per cycle at 2° C.; probe is stained in antibody solution for 10 minutes at 25° C.; probe is stained with the second aliquot of SAPE for 10 minutes at 25° C.; final wash is 15 cycles of 4 mixes per cycles at 30° C.; holds at 25° C. The plates are scanned using the Hewlett-Packard GeneArray© Scanner (Affymetrix).

### Example 4

Those skilled in the art can, by the exercise of ordinary skill, measure the mRNA or protein level for each of the two, three or more (preferably two to six) in a correlated set discovered to be diagnostic for leukemia subtype and. in reference to a standard, classify new cases of leukemia with respect to subtype. Such an analysis would be highly amenable to modern diagnostic “chip” technology and suitable for incorporation into a bedside diagnostic device.

For example, in reference to Appendix A, page i, cset 1 for AML, the expression level of Affymetrix designated genes Zyxin (GenBank Accession #X95735_at) and ELA2 Elastase 2, neutrophil (GenBank Accession #M27783_at) is diagnostic of AML. In this case, diagnosis of AML can be made if the relative expression level of each of these genes is high. Similarly, other csets in Appendix A provide diagnostic gene “signatures” or “fingerprints” of similar value.

[image]

[image]

[image]

[image]

[image]

[image]

[image]

[image]

[image]

[image]

[image]

[image]

[image]

[image]

[image]

[image]

[image]

[image]

[image]

It will be clear that the invention may be practiced otherwise than as particularly described in the foregoing description and examples.

Numerous modifications and variations of the present invention are possible in light of the above teachings and, therefore, within the scope of the appended claims, the invention may be practiced otherwise than as particularly described.

The entire disclosure of all publications (including patents, patent applications, journal articles, databases, GenBank entries, web sites, laboratory manuals, books, or other documents) cited herein are hereby incorporated by reference.

What is claimed is:

1. A method for diagnosing acute lymphoblastic leukemia (ALL), comprising:
(a) measuring the levels of gene expression of leukotriene C4 synthase (LTC4S) gene and Zyxin in a biological sample taken from a patient suspected of having ALL; and
(b) comparing the levels of gene expression in said biological sample with a standard sample, wherein low levels of expression are indicative of a diagnosis of ALL.

2. A method for diagnosing ALL, comprising:
(a) measuring the levels of gene expression of LYN V-yes-1 Yamaguchi sarcoma viral related oncogene homolog, PPGB Protective protein for beta-galactosidase, and Zyxin in a biological sample taken from a patient suspected of having ALL; and
(b) comparing the levels of gene expression in said biological sample with a standard sample, wherein low levels of expression are indicative of a diagnosis of ALL.

3. A method for determining a prognosis of a patient with AML, comprising:
(a) measuring the levels of gene expression of POU3F1 POU domain, class 3, transcription factor 1 and GB DEF=homeodomain protein HoxA9 mRNA in a biological sample taken from a patient with AML; and
(b) comparing the levels of gene expression in said biological sample with a standard sample, wherein medium-high levels of POU3F1 POU domain, class 3, transcription factor 1 and high levels of GB DEF=homeodomain protein HoxA9 mRNA, are indicative of a favorable prognosis.

4. A method for screening drugs which are useful for treating acute leukemia, comprising:
(a) administering to a cell culture a drug of interest;
(b) comparing the levels of gene expression of leukotriene C4 synthase (LTC4S) gene and/or Zyxin before administration of said drug with the levels of gene expression after administration of said drug, wherein a modulation of gene expression level after administration of the drug is indicative of a drug useful for treating acute leukemia.

5. A method for screening drugs which are useful for treating acute leukemia, comprising:
(a) administering to a cell culture a drug of interest; and
(b) comparing the levels of gene expression of LYN V-yes-1 Yamaguchi sarcoma viral related oncogene homolog, PPGB Protective protein for beta-galactosidase, and/or Zyxin before administration of said drug with the levels of gene expression after administration of said drug, wherein a modulation of gene expression level after administration of the drug is indicative of a drug useful for treating acute leukemia.

6. A kit for diagnosing ALL, comprising:
(a) a means for measuring gene expression of leukotriene C4 synthase (LTC4S) gene; and
(b) a means for measuring gene expression of Zyxin.

7. A kit for diagnosing ALL, comprising:
(a) a means for measuring gene expression of LYN V-yes-1 Yamaguchi sarcoma viral related oncogene homolog;
(b) a means for measuring gene expression of PPGB Protective protein for beta-galactosidase; and
(c) a means for measuring gene expression of Zyxin.

8. A method for screening drugs which are useful for treating acute leukemia, comprising:
(a) administering to a cell culture a drug of interest; and
(b) comparing the levels of gene expression of POU3F1 POU domain, class 3, transcription factor 1 and/or GB DEF=homeodomain protein HoxA9 mRNA in a biological sample taken from a patient with acute leukemia, wherein a modulation of gene expression level after administration of the drug is indicative of a drug useful for treating acute leukemia.

9. The use of gene expression levels of leukotriene C4 synthase (LTC4S) gene and Zyxin to diagnose ALL.

10. The use of gene expression levels of LYN V-yes-1 Yamaguchi sarcoma viral related oncogene homolog, PPGB Protective protein for beta-galactosidase, and Zyxin to diagnose ALL.

11. The use of gene expression levels of POU3F1 POU domain, class 3, transcription factor 1 and GB DEF=homeodomain protein HoxA9 mRNA for the prognosis of AML.

12. A method for diagnosing acute myeloid leukemia (AML), comprising:
(a) measuring the levels of gene expression of Zyxin and ELA2 Elastase 2, neutrophil, in a biological sample taken from a patient suspected of having AML; and
(b) comparing the levels of gene expression in said biological sample with a standard sample, wherein high levels of expression are indicative of a diagnosis of AML.
